(19) United States
(12) Patent Application Publication
(10) Pub. No.: US 2024/0207656 A1
(43) Pub. Date: Jun. 27, 2024

(54) METHODS OF IMPROVING A COGNITIVE DISORDER BY NEUROMODULATION OF A PULVINAR NUCLEUS

(71) Applicant: West Virginia University Board of Governors on behalf of West Virginia University, Morgantown, WV (US)

(72) Inventors: Ali R. Rezai, Morgantown, WV (US); Victor Finomore, Morgantown, WV (US)

(21) Appl. No.: 18/398,011

(22) Filed: Dec. 27, 2023

Related U.S. Application Data
(60) Provisional application No. 63/435,456, filed on Dec. 27, 2022.

(51) Int. Cl. A61N 7/02; A61N 1/36; A61N 2/00

(52) U.S. Cl. CPC A61N 7/02; A61N 1/36082; A61N 2/006; A61N 2007/0026

(57) ABSTRACT

A method of improving a cognitive disorder in a patient suffering therefrom is provided. The method includes diagnosing the patient with the cognitive disorder and applying a neuromodulation treatment, such as focused ultrasound, to a pulvinar nucleus of the patient. The method can include acquiring a clinical parameter associated with the patient and applying the neuromodulation treatment or adjusting application of the neuromodulation treatment to the pulvinar nucleus of the patient based on the clinical parameter.

### RELATED APPLICATION

This application is related to U.S. Provisional Application Ser. No. 63/435,456, filed on Dec. 27, 2022 and entitled “SCREENING, MONITORING, AND TREATMENT OF COGNITIVE DISORDERS,” which is hereby incorporated by reference.

### TECHNICAL FIELD

This disclosure relates generally to the field of medical systems, and more particularly to screening, monitoring, and treatment of cognitive disorders.

### BACKGROUND

Cognitive disorders occur when nerve cells in the brain or peripheral nervous system lose function over time and ultimately die. Although treatment may help relieve some of the physical or mental symptoms associated with cognitive disorders, there is currently no effective treatment for arresting the process of these disorders. The likelihood of developing a cognitive disorder rises dramatically with age, and it is expected that more people may be affected by cognitive disorders as life expectancy increases. Unfortunately, detection and monitoring of these conditions, particularly in the early stages, is currently difficult.

### SUMMARY OF THE INVENTION

In accordance with an aspect of the invention, a method is provided for generating a value representing one of a risk and a progression of a cognitive disorder. The method includes acquiring a first image, representing a brain of a patient, from a first imaging system and acquiring a second image, representing one of a retina, an optic nerve, and a vasculature associated with one of the optic nerve and the retina of the patient, from a second imaging system. A representation of each of the first image and the second image are provided to a machine learning model. The value is generated at the machine learning model from the representation of the first image and the representation of the second image, and the patient is assigned to one of a plurality of intervention classes according to the generated value. An intervention is provided to the patient according to the assigned intervention class.

In accordance with another aspect of the invention, a system is provided for generating a value representing one of a risk and a progression of one or more cognitive disorders. The system includes a processor and a non-transitory computer readable medium storing machine-readable instructions executable by the processor to provide an imager interface that acquires a first image, representing a brain of a patient, from a first imaging system and a second image, representing one of a retina, an optic nerve, and a vasculature associated with one of the retina and the optic nerve of the patient, from a second imaging system. A machine learning model generates the value from a representation of the first image and a representation of the second image. An assisted decision making module assigns the patient to one of a plurality of intervention classes according to the generated value, and a display displays the assigned intervention class to a user.

In accordance with a further aspect of the invention, a method includes diagnosing a patient with a cognitive disorder and applying a first focused ultrasound treatment to the patient at a location of interest. A first clinical parameter for the patient is acquired either during or immediately after the first focused ultrasound treatment. The first clinical parameter represents at least one of eye tracking data, eye movement, pupil size, and a change in pupil size. A second clinical parameter for the patient is acquired between five hours and five days after the first focused ultrasound treatment. A third clinical parameter is acquired for the patient more than five days after the first focused ultrasound treatment from an acquired image representing one of a retina, an optic nerve, and a vasculature associated with one of the optic nerve and the retina of the patient from an imaging system. A value representing progression of a cognitive disorder is generated at the machine learning model from the first clinical parameter, the second clinical parameter, and the third clinical parameter. A second focused ultrasound treatment is provided to the patient according to the generated value.

In accordance with another aspect of the invention, a method of improving a cognitive disorder in a patient suffering therefrom includes diagnosing the patient with a cognitive disorder, applying focused ultrasound treatment to a pulvinar nucleus of the patient, and improving the cognitive disorder in the patient.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 illustrates one example of a system for diagnosis and monitoring of cognitive disorders;

FIG. 2 illustrates another example of a system for diagnosis and monitoring of cognitive disorders;

FIG. 3 illustrates a system for targeting neuromodulation for treatment or diagnosis of cognitive disorders to a specific region of the brain of a patient;

FIG. 4 illustrates a system for evaluating the effects of neuromodulation on one or more patients;

FIG. 5 illustrates a method for generating a value representing a risk and/or progression of one or more cognitive disorders;

FIG. 6 illustrates another method for generating a value representing a risk and/or progression of one or more cognitive disorders;

FIG. 7 illustrates a method for obtaining chronic feedback for a neuromodulation treatment;

FIG. 8 illustrates a method of improving a cognitive disorder in a patient suffering therefrom; and

FIG. 9 illustrates a method for providing ultrasound treatment;

FIG. 10 is a schematic block diagram illustrating an exemplary system of hardware components capable of implementing examples of the systems and methods disclosed in FIGS. 1-9.

### DETAILED DESCRIPTION

Various examples of the systems and methods described herein utilize images of the brain, as well as one or more of the retina, the optic nerve, and the associated vasculature, as assessed, for example, by OCT, OCT-A, and fundus photography to diagnose, monitor, and guide treatment of cognitive disorders. These images, generally used in concert with various clinical parameters associated with the patient can be utilized at a predictive model, implemented for example, as a machine learning model, to detect or monitor neurodegeneration generally or the presence or progression of a specific disorder. In one example, other parameters associated with the eye, specifically the pupil, can be used in concert with this information. For example, the machine learning model can receive one or more parameters representing eye tracking data, eye movement, pupil size, or a change in pupil size. Accordingly, the system provides early detection and precise monitoring of such disorders.

A “neuromodulation technique,” or “neuromodulation” as described herein, is any suitable technique that applies localized energy (or other mode of neuromodulation) to the brain for the purpose of modulating neural activity to alleviate and/or improve the symptoms of a cognitive disorder, such as hallucinations, deficits in attention, memory, executive function, sensory processing, visual spatial function, and visual processing, and similar symptoms, and includes techniques such as, for example, focused ultrasound, electrical stimulation, such as superficial or deep brain stimulation, transcranial magnetic stimulation, or application of pulses of electromagnetic radiation. Other modes of neuromodulation include application of light, radiation, pressure, heat, and cold.

As used herein, a “clinical parameter” is any value representing a patient that is relevant to the patients' risk of a cognitive disorder or a progression of a cognitive disorder. Clinical parameters can include values measured by clinicians in a clinical environment, values measured outside of a clinical environment by one or more wearable or portable devices, or values retrieved from an electronic health records (EHR) interface and/or other available databases. It will be appreciated that the clinical parameter can be, for example, a physiological, cognitive, behavioral, psychosocial or anatomical parameter associated with the patient.

As used herein, a “predictive model” is a mathematical model or machine learning model that either predicts a future state of a parameter or estimates a current state of a parameter that cannot be directly measured.

As used herein, a “categorical value” is a value that can be represented by one of a number of discrete possibilities, which may or may not have a meaningful ordinal ranking. A “continuous value,” as used herein, is a value that can take on any of a number of numerical values within a range. It will be appreciated that, in a practical application, values are expressed in a finite number of significant digits, and thus a “continuous value” can be limited to a number of discrete values within the range.

As used herein, data is provided from a first system to a second system when it is either provided directly from the first system to the second system, for example, via a local bus connection, or stored in a local or remote non-transitory memory by the first system for later retrieval by the second system. Accordingly in some implementations, the first system and the second system can be located remotely and in communication only through an intermediate medium connected to at least one of the systems by a network connection.

A “cognitive disorder,” as used herein is a pathological condition in which the patient exhibits compromised cognitive function due to the dysfunction or loss of neurons and/or other nervous system components. Cognitive function includes general intellectual function, basic attention, complex attention (working memory), executive function, memory (visual and verbal), language, visio-constructional function, and visio-spatial construction. As such, non-limiting examples of cognitive functions include general intellectual function; sensory processing, including processing of all sensory input into context; attention, such as basic attention, the ability to monitor and direct attention, and the flexible allocation of attentional resources; working memory or divided attention which refers to a limited-capacity memory system in which information that is the immediate focus of attention can be temporarily held and manipulated (such as, for example, being able to simultaneously maintain two trains of thought in a flexible manner); executive functions which include, for example, planning, problem-solving skills, intentional and self-directed behavior, organizational skills, goal-directed behavior, the ability to generate multiple response alternatives, and maintenance of a conceptual set (i.e. the ability to maintain (or not lose) set or track of what one is doing); the ability to evaluate and modify behavior in response to feedback; verbal and visual memory, or the ability to retain and store new information for future use; visuo-spatial skills, such as judging how lines are oriented or discerning spatial relationships and patterns; visuospatial function; higher order processing of visual input; visuo-constructional skills including two-dimensional construction skills (such as, for example, drawing or completing puzzles) and three-dimensional constructional skills (such as, for example, arranging blocks to match a design); language such as confrontation naming (such as, for example, naming specific words on demand, such as when shown a picture of the object), word fluency or generating a nonredundant list of words that belong to a specific category; and combinations thereof.

Examples include Alzheimer's disease and related dementias, developmental disorders, such as autism, and other cognitive disorders such as schizophrenia, mild cognitive impairment (MCI), Lewy body dementia, frontotemporal dementia, vascular dementia, Parkinson's dementia, Chronic traumatic encephalopathy, Huntington's disease, and multi system atrophy, corticobasal degeneration, striatonigral disease, multiple sclerosis, conditions where the patient has a beta-amyloid protein, a tau protein, and/or another biomarker of a neurodegenerative disorder, and combinations thereof.

“Registration” of two or more images includes any process that assigns relative locations between pixels or multi-pixel features across two or more images. This assignation can be represented, for example, via an explicit transformation model between two or more images or via feature matching techniques that identify common structural features across two images.

An “average,” as used herein, can be any measure of central tendency, including but not limited to, an arithmetic mean, a geometric mean, a median, and a mode. It will be appreciated that, where a mean for a set of values is used as the average, the mean can be taken from a subset of the set of values to eliminate outliers within the set of values. For example, values between the fifth and the ninety-fifth percentile can be used to generate the mean.

An “intensity profile,” as used herein, represents a spatial variation in the intensity of localized energy provided to the brain. An intensity profile can include a variance between two sides of a region provided with the localized energy or a more complex spatial variation of the energy.

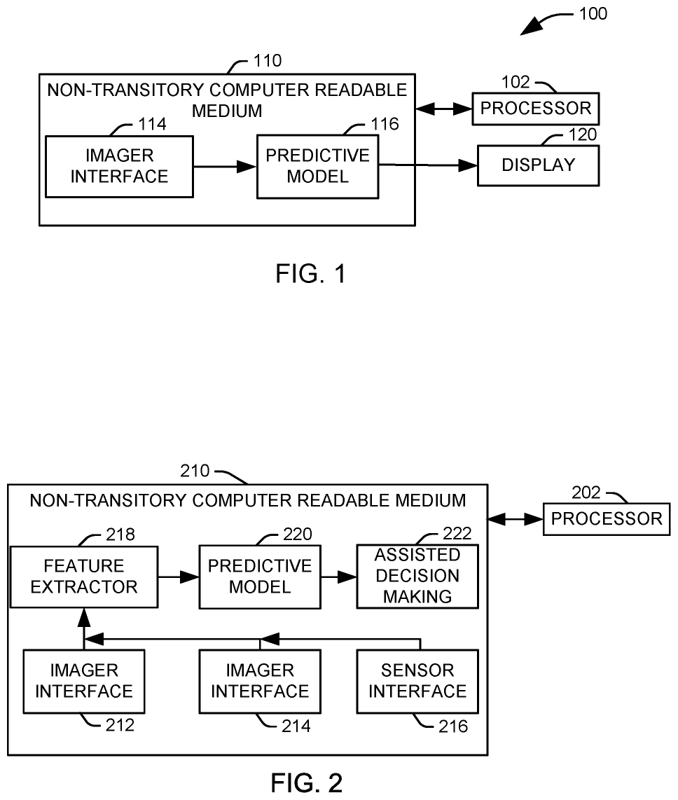

FIG. 1 illustrates one example of a system 100 for diagnosis and monitoring of cognitive disorders. The system 100 includes a processor 102 and a non-transitory computer readable medium 110 that stores executable instructions for receiving data representing a patient from a plurality of sources and determining a clinical parameter representing a likelihood that the patient has or will develop a cognitive disorder. The executable instructions include an imaging interface 112 that receives a first image, representing a structure or connectivity of a brain of the patient, and a second image, representing a one of the retina, the optic nerve, and the associated vasculature of the patient, from respective imaging systems (not shown). In one implementation, the first image is a T1 magnetic resonance imaging (MRI) image representing a structure of the brain. In other implementations, the first image can be a diffusion tensor imaging (DTI) image generated using an MRI imager, a position emission tomography (PET) image acquired using glucose tagged with radioactive fluorine or a tracer for beta-amyloid or the tau protein. The second image can be any image from which the retina, optical nerve, or associated vasculature can be extracted, for example, an optical coherence tomography (OCT) image, an OCT angiography image, and an image acquired via fundus photography. In some examples, the imager interface 112 can also image a pupil of the patient to provide a parameter representing at least one of eye tracking data, eye movement, pupil size, and a change in pupil size.

The imaging interface 112 can include appropriate software components for communicating with an imaging system (not shown) or repository of stored images (not shown) over a network via a network interface (not shown) or via a bus connection. In some implementations, the imaging interface 112 can segments the first image into a plurality of subregions of the brain, such that each of at least a portion of the pixels or voxels comprising the image are associated with one of the plurality of subregions. The identified subregions can include, for example, a frontal pole, a temporal pole, a superior frontal region, a medial orbito-frontal region, a caudal anterior cingulate, a rostral anterior cingulate, an entorhinal region, a parahippocampal region, a peri-calcarine region, a lingual region, a cuneus region, an isthmus region, a pre-cuneus region, a paracentral lobule, and a fusiform region. In one example, the imaging interface 112 registers the first image to a standard atlas to provide the segmentation. In another implementation, a convolutional neural network, trained on a plurality of annotated image samples, can be used to provide the segmented image. One example of such a system can be found in 3D Whole Brain Segmentation using Spatially Localized Atlas Network Tiles, by Huo et al. (available at https://doi.org/10.48550/arxiv.1903.12152), which is hereby incorporated by reference in its entirety. Where multiple images representing the brain taken in different imaging modalities are provided, the imager interface 112 can register the images with one another. For example, an image representing the structure can be registered with an image representing brain connectivity such that the location of nodes within the connectome within the brain is known.

A predictive model 116 receives a representation of the first image and a representation of the second image and generates a value representing a clinical property associated with one or more cognitive disorders for the patient. For example, the generated value is a categorical or continuous value that represents a likelihood that the patient currently has a specific cognitive disorder or a cognitive disorder generally. In another example, the generated value is a categorical or continuous value that represents a likelihood that the patient will develop a specific cognitive disorder or a cognitive disorder generally. In a yet another, the generated value is a categorical or continuous value that represents a likelihood that the patient will respond to a specific treatment for a cognitive disorder or to treatment of a cognitive disorder generally. In a further example, the generated value is a categorical value that represents an expected best treatment for a cognitive disorder for the patient. In a still further example, the generated value is a continuous or categorical value that represents a progression of a cognitive disorder for the patient, either in general, or specifically in response to an applied treatment.

The representation of each of the first image and the second image provided to the predictive model can include any of the images themselves, represented as chromaticity values from the pixels or voxels comprising the image, images or masks derived from the images, or sets of numerical features extracted from the image. For example, the representation of the first image can be any of a representation of a cortical profile of the brain, a representation of a vasculature of the brain, a representation of a B-amyloid profile of the brain, and a representation of a connectivity of the brain from the first image. It will be appreciated that the predictive model 116 can also receive clinical parameters representing the patient, for example, measured via one or more sensors and/or retrieved from a medical health records database, such that the value representing a clinical property associated with one or more cognitive disorders for the patient is calculated from the representation of the first image, the representation of the second image, and the clinical parameters.

The predictive model 116 can utilize one or more pattern recognition algorithms, implemented, for example, as classification and regression models, each of which analyze the provided data to assign the value to the user. Where multiple classification and regression models are used, the predictive model can include an arbitration element can be utilized to provide a coherent result from the various algorithms. Depending on the outputs of the various models, the arbitration element can simply select a class from a model having a highest confidence, select a plurality of classes from all models meeting a threshold confidence, select a class via a voting process among the models, or assign a numerical parameter based on the outputs of the multiple models. Alternatively, the arbitration element can itself be implemented as a classification model that receives the outputs of the other models as features and generates one or more output classes for the patient.

The predictive model, as well as any constituent models, can be trained on training data associated with known patient outcomes. Training data can include, for example, the representations of the first and second images for a plurality of patients each labeled with a known outcome for the patient. The training process of the predictive model will vary with its implementation, but training generally involves a statistical aggregation of training data into one or more parameters associated with the output classes. For rule-based models, such as decision trees, domain knowledge, for example, as provided by one or more human experts, can be used in place of or to supplement training data in selecting rules for classifying a user using the input data. Any of a variety of techniques can be utilized for the models, including support vector machines, regression models, self-organized maps, k-nearest neighbor classification or regression, fuzzy logic systems, data fusion processes, boosting and bagging methods, rule-based systems, or artificial neural networks. Regardless of the specific model employed, the categorical or continuous value generated at the predictive model 116 can be provided to a user at the display 120 via a user interface or stored on the non-transitory computer readable medium 110, for example, in an electronic medical record associated with the patient.

FIG. 2 illustrates one example of a system for diagnosis and monitoring of cognitive disorders. The system 200 includes a processor 202 and a non-transitory computer readable medium 210 that stores executable instructions for retrieving and diagnosis and monitoring of cognitive disorders. The executable instructions include a first imager interface 212 that receives at least one image representing a brain of a patient. In one example, the first imager interface 212 is configured to receive and condition magnetic resonance imaging (MRI) images, and the at least one image includes a first image representing a structure of the brain, and a second image, representing a connectivity of the brain. In one implementation, the first image is a T1 magnetic resonance imaging (MRI) image, and the second image is a diffusion tensor imaging (DTI) image generated using an MRI imager. The first imager interface 212 can also receive functional images, for example, from a same or a different MRI imager or a positron emission tomography (PET) imager, representing activity within the brain. The functional images can be taken either during a resting state or while the patient is performing a task. In some implementations, multiple images of the brain can be registered to one another at the first imager interface 212 via a registration process. A second imager interface 214 receives at least one image representing a one of a retina, an optic nerve, and an associated vasculature of a patient from an associated imaging system. For example, the second imager interface 214 can receive images from one or more of an optical coherence tomography (OCT) imager, for example, as an OCT angiography image, a camera used for fundus photography, and a camera used for fluorescein angiography images. Each of the first imager interface 212 and the second imager interface 214 can include appropriate software components for communicating with an associated imaging system (not shown) or repository of stored images (not shown) over a network via a network interface (not shown) or via a bus connection.

A sensor interface 216 can receive clinical parameters measured by one or more wearable or portable devices as well as clinical parameters retrieved from an electronic health records (EHR) interface and/or other available databases via a network interface (not shown). These parameters can include, for example, employment information (e.g., title, department, shift), age, sex, home zip code, genomic data, nutritional information, medication intake, household information (e.g., type of home, number and age of residents), social and psychosocial data, consumer spending and profiles, financial data, food safety information, the presence or absence of physical abuse, and relevant medical history.

Clinical parameters useful for screening for cognitive disorders can include at least physiological, cognitive, motor/musculoskeletal, sensory, sleep, biomarkers, and behavioral parameters. Table I provides non-limiting examples of physiological parameters that can be measured and exemplary tests, devices, and methods, to measure the physiological parameters.

TABLE I

| | Exemplary Devices and Methods to Measure |
|---|---|
| Physiological Parameter | Physiological Parameters |
| Brain Activity | Electroencephalogram, Magnetic Resonance Imaging, |
| | including functional Magnetic Resonance Imaging |
| | (fMRI), PET, SPECT, MEG, near-infrared spectroscopy, |
| | functional near-infrared spectroscopy, and other brain |
| | imaging modalities looking at electrical, blood flow, |
| | neurotransmitter, and metabolic function |
| Heart rate | Electrocardiogram and Photoplethysmogram |
| Heart rate variability | Electrocardiogram, Photoplethysmogram |
| Eye tracking | Pupillometry, including tracking saccades, fixations, and |
| | pupil size (e.g., dilation) |
| Perspiration | Perspiration sensor |
| Blood pressure | Sphygmomanometer |
| Body temperature | Thermometer, infrared thermography |
| Blood oxygen saturation | Pulse oximeter/accelerometer |
| and respiratory rate | |
| Skin conductivity | Electrodermal activity |
| Facial emotions | Camera or EMG based sensors for emotion and |
| | wellness |
| Sympathetic and | Derived from the above measurements |
| parasympathetic tone | |

The physiological parameters can be measured via wearable or implantable devices as well as self-reporting by the user via applications in a mobile device, which facilitates measuring these physiological parameters in a naturalistic, non-clinical setting. For example, a smart watch, ring, or patch can be used to measure the user's heart rate, heart rate variability, body temperature, blood oxygen saturation, movement, and sleep. These values can also be subject to a diurnal analysis to estimate variability and reviewed in view of expected changes due to biological rhythms, as well as deviations from an expected pattern of biological rhythms. For example, the biological rhythms of a user can be tracked for a predetermined period (e.g., ten days), to establish a normal pattern of biological rhythms. Oscillations in biological rhythms can be detected as departures from this established pattern. Table II provides non-limiting examples of cognitive parameters that are gamified and that can be measured and exemplary methods and tests/tasks to measure such cognitive parameters. The cognitive parameters can be assessed by a battery of cognitive tests that measure, for example, executive function, decision making, working memory, attention, and fatigue.

TABLE II

| | Exemplary Tests and Methods to Measure |
|---|---|
| Cognitive Parameter | Cognitive Parameters |
| Temporal discounting | Kirby Delay Discounting Task |
| Alertness and fatigue | Psychomotor Vigilance Task |
| Focused attention and | Erikson Flanker Task |
| response inhibition | |
| Working memory | N-Back Task, episodic memory task, spatial navigation |
| | task, word list, picture naming |
| Attentional bias towards | Dot-Probe Task |
| emotional cues | |
| Visual-spatial | Numerosity tasks, change detection, |
| Perceptual motor | Choice reaction time, object manipulation, |
| Inflexible persistence | Wisconsin Card Sorting Task |
| Decision making | lowa Gambling Task |
| Risk taking behavior | Balloon Analogue Risk Task |
| Inhibitory control | Anti-Saccade Task |
| Sustained attention | Sustained Attention |
| Executive function | Task Shifting or Set Shifting Task |
| Neuropsychological | Repeatable Battery for the Assessment of |
| assessments | Neuropsychological Status, Mini-Mental State |
| | Examination, Neuropsychiatric inventory- |
| | questionnaire, Alzheimer's Disease Cooperative Study- |
| | activities of daily living inventory, |

These cognitive tests can be administered in a clinical/laboratory setting or in a naturalistic, non-clinical setting such as when the user is at home, work, or other non-clinical setting. A smart device, such as a smartphone, tablet, or smart watch, can facilitate measuring these cognitive parameters in a naturalistic, non-clinical setting and ecological momentary assessments in a natural environment. For example, the Erikson Flanker, N-Back and Psychomotor Vigilance Tasks can be taken via an application on a smart phone, tablet, or smart watch.

TABLE III provides non-limiting examples of parameters associated with movement and activity of the user, referred to herein alternatively for ease of reference as “motor parameters,” that can be measured and exemplary tests, devices, and methods. The use of portable monitoring, physiological sensing, and portable computing devices allows the motor parameters to be measured. Using embedded accelerometer, GPS, and cameras, the user's movements can be captured and quantified to generate clinical parameters.

TABLE III

| Motor/Musculoskeletal | Exemplary Tests and Methods to Measure |
|---|---|
| Parameter | Motor/Musculoskeletal Parameters |
| Activity level | Daily movement total, time of activities, from wearable |
| | accelerometer, steps, Motion Capture data, gait |
| | analysis, GPS, deviation from established geolocation |
| | patterns, force plates |
| Gait analysis | Gait mat, camera, force plats |
| Range of motion | Motion capture, camera, |

TABLE IV provides non-limiting examples of parameters associated with sensory acuity of the user, referred to herein alternatively for ease of reference as “sensory parameters,” that can be measured and exemplary tests, devices, and methods.

TABLE IV

| | Exemplary Tests and Methods to Measure sensory |
|---|---|
| Sensory Parameter | Parameters |
| Vision | Visual acuity test, visual field tests, eye tracking, EMG, |
| | blink reflex test |
| Hearing | Hearing tests |
| Touch | Two-point discrimination, frey filament |
| Smell/taste | |
| Vestibular | Vestibula function test |

TABLE V provides non-limiting examples of parameters associated with a sleep quantity and quality of the user, referred to herein alternatively for ease of reference as “sleep parameters,” that can be measured and exemplary tests, devices, and methods.

TABLE V

| | Exemplary Tests and Methods to Measure Sleep |
|---|---|
| Sleep Parameter | Parameters |
| Sleep from wearables | Sleep onset & offset, sleep quality, sleep quantity, from |
| | wearable accelerometer, temperature, and PPG, |
| Sleep Questions | Pittsburg Sleep Quality Index, Functional Outcomes of |
| | Sleep Questionnaire, Fatigue Severity Scale, Epworth |
| | Sleepiness Scale |
| Devices | Polysomnography; ultrasound, camera, bed sensors |
| Circadian Rhythm | Light sensors, actigraphy, serum levels, core body |
| | temperature |

TABLE VI provides non-limiting examples of parameters extracted by locating biomarkers associates with the user, referred to herein alternatively for ease of reference as “biomarker parameters,” that can be measured and exemplary tests, devices, and methods. Biomarkers can also include imaging and physiological biomarkers related to a cognitive disorder and improvement or worsening of a cognitive disorder.

TABLE VI

| | Exemplary Tests and Methods to Measure |
|---|---|
| Biomarkers Parameter | Biomarkers Parameters |
| Genetic biomarkers | Genetic testing |
| Immune biomarkers including | Blood, saliva, and/or urine tests |
| TNF-alpha, immune alteration | |
| (e.g., ILs), oxidative stress, | |
| and hormones (e.g., cortisol) | |
| Cerebrospinal fluid and /or | Beta-amyloid 42, tau, phosphor-tau |
| blood biomarkers | |

Table VII provides non-limiting examples of psychosocial and behavioral parameters, referred to herein alternatively for ease of reference as “psychosocial parameters,” that can be measured and exemplary tests, devices, and methods.

TABLE VII

| Psychosocial or | Exemplary Tests and Methods to Measure |
|---|---|
| Behavioral Parameter | Psychosocial or Behavioral Parameters |
| Symptom log | Presence of specific symptoms (i.e., fever, headache, |
| | cough, loss of smell) |
| Medical Records | Medical history, prescriptions, setting for treatment |
| | devices such as spinal cord stimulator, imaging data |
| Wellness Rating | Visual Analog Scale, Defense & Veterans wellness rating |
| | scale, wellness scale, Wellness Assessment screening |
| | tool and outcomes registry |
| Burnout | Burnout inventory or similar |
| Physical, Mental, and Social | User-Reported Outcomes Measurement Information |
| Health | System (PROMIS), Quality of Life Questionnaire |
| Depression | Hamilton Depression Rating Scale, Geriatric depression |
| | scale, Columbia suicide rating scale |
| Anxiety | Hamilton Anxiety Rating Scale |
| Mania | Snaith-Hamilton Pleasure Scale |
| Mood/Catastrophizing Scale | Profile of Mood States; Positive Affect Negative Affect |
| | Schedule |
| Affect | Positive Affect Negative Affect Schedule |
| Impulsivity | Barratt Impulsiveness Scale |
| Adverse Childhood | Childhood trauma |
| Experiences | |
| Daily Activities | Exposure, risk taking |
| Daily Workload and Stress | NASA Task Load Index, Perceived Stress Scale (PSS), |
| | Social Readjustment Rating Scale (SRRS) |
| Social Determents of Health | Social determents of health questionnaire |

The behavioral and psychosocial parameters can measure the user's functionality as well as subjective/self-reporting questionnaires. The subjective/self-reporting questionnaires can be collected in a clinical/laboratory setting or in a naturalistic, in the wild, non-clinical setting such as when the user is at home, work, or other non-clinical setting. A smart device, such as a smartphone, tablet, or personal computer can be used to administer the subjective/self-reporting questionnaires. Using embedded accelerometers and cameras, these smart devices can also be used to capture the facial expression analysis to analyze the user's facial expressions that could indicate mood, anxiety, depression, agitation, and fatigue.

A feature extractor 218 extracts a set of features from one or more of the clinical parameters, the image or images from the first imager interface 212, and the image or images from the second imager interface 214. In one example, the feature extractor 218 can extract various biometric parameters from the brain images from the first imager interface 212, including parameters related to connectivity, gyrification, volume, vascular load, networks, a white matter load, a grey/white mater ratio, cortical thickness, sulcal depth and other measurements. Images of the brain can also be segmented to determine these parameters for specific regions of the brain. Similarly, numerical parameters can be extracted from the images of the eye, including a volume, thickness, or texture of the retina or individual retinal layers at one or more locations or an average thickness of the retina or one or more layers across locations, values representing the vascular pattern and density of the retina or individual layers of the retina, a size of the foveal avascular zone, a height, width, or volume of the optic chiasm, a height, width, or volume of the intraorbital optic nerve, a height, width, or volume of the intracranial optic nerve, a total area of the vasculature in the image. The feature extractor 218 can also receive images of a pupil of the patient, and generate parameters representing one or more of eye tracking data, eye movement, pupil size, and a change in pupil size. Additionally or alternatively, the chromaticity values associated with the individual pixels or voxels within each image can be provided directly as features, with a predictive model 220 applying various convolutional kernels to the image to generate features from the raw values from the pixel or voxels.

The feature extractor 218 can also determine categorical and continuous features representing the clinical parameters. In one example, the features can include descriptive statistics, such as measures of central tendency (e.g., median, mode, arithmetic mean, or geometric mean) and measures of deviation (e.g., range, interquartile range, variance, standard deviation, etc.) of time series of the monitored parameters, as well as the time series themselves. Specifically, the feature set provided to the predictive model 220 can include, for at least one parameter, either two values representing the value for the parameter at different times or a single value, such as a measure of central tendency or a measure of deviation which represents values for the parameter across a plurality of times.

In other examples, the features can represent departures of the user from an established pattern for the parameters. For example, values of a given parameter can be tracked overtime, and measures of central tendency can be established, either overall or for particular time periods. The collected features can represent a departure of a given parameter from the measure of central tendency. For example, changes in the activity level of the user, measured by either or both of kinematic sensors and global positioning system (GPS) tracking can be used as a feature. Additional elements of monitoring can include the monitoring of the user's compliance with the use of a smart phone, TV, portable device, a portable device. For example, a user may be sent messages by the system inquiring on their wellness level, general mood, or the status of any other clinical parameter on the portable computing device. A measure of compliance can be determined according to the percentage of these messages to which the user responds via the user interface on the portable computing device.

In one implementation, the feature extractor 218 can perform a wavelet transform on a time series of values for one or more parameters to provide a set of wavelet coefficients. It will be appreciated that the wavelet transform used herein is two-dimensional, such that the coefficients can be envisioned as a two-dimensional array across time and either frequency or scale.

For a given time series of parameters, xᵢ, the wavelet coefficients, Wₐ(n), produced in a wavelet decomposition can be defined as:

$${W}_{a} ( n ) = {a}^{- 1} {\sum }_{i = 1}^{M} {x}_{i} ψ ( \frac{i - n}{a} ) \qquad Eq . 3$$

wherein ψ is the wavelet function, Mis the length of the time series, and a and n define the coefficient computation locations.

The predictive model 220 can utilize one or more pattern recognition algorithms, each of which analyze the extracted features or a subset of the extracted features to assign a continuous or categorical value to the user representing a risk or a progression of cognitive disorder. In one example, the predictive model 220 can assign a continuous parameter that corresponds to a likelihood that that user has or is at high risk for a specific cognitive disorder, a likelihood that that is at high risk for cognitive disorders generally, a likelihood that the user is experiencing the effects of aging, a likelihood that the user is experiencing an onset of dementia, a likelihood that the user has or will develop a cognitive disorder, a likelihood that the user will experience an intensifying of symptoms of a cognitive disorder, a current or predicted response to treatment for a cognitive disorder, or a progression of an existing cognitive disorder. In another example, the predictive model 220 can assign a categorical parameter that corresponds to ranges of the likelihoods described above, the presence or predicted presence of a specific cognitive disorder, categories representing changes in symptoms associated with a cognitive disorder (e.g., “improving”, “stable, “worsening”), categories representing a current or predicted response to treatment, or categories indicating that a particular action should be suggested to the user. The generated parameter can be stored in a non-transitory computer readable medium, for example, as part of a record in an electronic health records database, or used to suggest a treatment or course of action to the user.

Where multiple classification or regression models are used, an arbitration element can be utilized to provide a coherent result from the plurality of models. The training process of a given classifier will vary with its implementation, but training generally involves a statistical aggregation of training data into one or more parameters associated with the output class. The training process can be accomplished on a remote system and/or on a local device, such as a wearable or portable device. The training process can be achieved in a federated or non-federated fashion. For rule-based models, such as decision trees, domain knowledge, for example, as provided by one or more human experts or extracted from existing research data, can be used in place of or to supplement training data in selecting rules for classifying a user using the extracted features. Any of a variety of techniques can be utilized for the classification algorithm, including support vector machines, regression models, self-organized maps, fuzzy logic systems, data fusion processes, boosting and bagging methods, rule-based systems, or artificial neural networks.

Federated learning (aka collaborative learning) is a predictive technique that trains an algorithm across multiple decentralized edge devices or servers holding local data samples, without exchanging their data samples. This approach stands in contrast to traditional centralized predictive techniques where all data samples are uploaded to one server, as well as to more classical decentralized approaches which assume that local data samples are identically distributed. Federated learning enables multiple actors to build a common, robust predictive model without sharing data, thus addressing critical issues such as data privacy, data security, data access rights, and access to heterogeneous data. Its applications are spread over a number of industries including defense, telecommunications, IoT, or pharmaceutics.

An SVM classifier can utilize a plurality of functions, referred to as hyperplanes, to conceptually divide boundaries in the N-dimensional feature space, where each of the N dimensions represents one associated feature of the feature vector. The boundaries define a range of feature values associated with each class. Accordingly, an output class and an associated confidence value can be determined for a given input feature vector according to its position in feature space relative to the boundaries. In one implementation, the SVM can be implemented via a kernel method using a linear or non-linear kernel.

An ANN classifier comprises a plurality of nodes having a plurality of interconnections. The values from the feature vector are provided to a plurality of input nodes. The input nodes each provide these input values to layers of one or more intermediate nodes. A given intermediate node receives one or more output values from previous nodes. The received values are weighted according to a series of weights established during the training of the classifier. An intermediate node translates its received values into a single output according to a transfer function at the node. For example, the intermediate node can sum the received values and subject the sum to a binary step function. A final layer of nodes provides the confidence values for the output classes of the ANN, with each node having an associated value representing a confidence for one of the associated output classes of the classifier. Another example is utilizing an autoencoder to detect outliers in clinical parameters as an anomaly detector to identify when various parameters are outside their normal range for an individual.

Many ANN classifiers are fully connected and feedforward. A convolutional neural network, however, includes convolutional layers in which nodes from a previous layer are only connected to a subset of the nodes in the convolutional layer. Recurrent neural networks are a class of neural networks in which connections between nodes form a directed graph along a temporal sequence. Unlike a feedforward network, recurrent neural networks can incorporate feedback from states caused by earlier inputs, such that an output of the recurrent neural network for a given input can be a function of not only the input but one or more previous inputs. As an example, Long Short-Term Memory (LSTM) networks are a modified version of recurrent neural networks, which makes it easier to remember past data in memory.

A rule-based classifier applies a set of logical rules to the extracted features to select an output class. Generally, the rules are applied in order, with the logical result at each step influencing the analysis at later steps. The specific rules and their sequence can be determined from any or all of training data, analogical reasoning from previous cases, or existing domain knowledge. One example of a rule-based classifier is a decision tree algorithm, in which the values of features in a feature set are compared to corresponding threshold in a hierarchical tree structure to select a class for the feature vector. A random forest classifier is a modification of the decision tree algorithm using a bootstrap aggregating, or “bagging” approach. In this approach, multiple decision trees are trained on random samples of the training set, and an average (e.g., mean, median, or mode) result across the plurality of decision trees is returned. For a classification task, the result from each tree would be categorical, and thus a modal outcome can be used.

In one example, the predictive model 220 can include multiple models that accept different sets of inputs and provide values representing different aspects of a patient's risk or progression of cognitive disorder. In one example, a first model uses only clinical parameters received at wearable and portable devices associated with the user and data from the patient's electronic medical record to determine if it is likely that the patient may be in the early stages of a cognitive disorder. When the first model determines that the patient may be developing a cognitive disorder, the patient can be instructed to visit a physician to have appropriate imaging performed to provide images of the patient's brain and eye for a second model that provides a more accurate assessment of the patient's risk of a cognitive disorder.

Once the value representing the patient's risk or progression of a cognitive disorder is determined, the patient can be assigned to an intervention class at an assisted decision-making component 222. An “intervention class”, as used herein, is a group of patients that are likely to respond well to a particular treatment, class of treatments, or non-treatment. For example, if the patient is determined to be at a low risk, the patient can be assigned various non-clinical interventions, such as changes in diet or sleep or assignment of brain exercises to help support healthy aging, or even no intervention. Patients at moderate risk can be assigned a therapeutic agent, such as medication or antibody treatments, and/or referred to rehabilitation, a clinical trial, family planning, or a support group. If the patient is determined to be at high risk for a disorder or with mild or moderate cognitive disorders, they can be assigned neuromodulation, such as deep brain stimulation or focused ultrasound. Patients with more severe neurodegeneration can be assigned to a more intensive treatment, such as the application of drugs or antibodies with targeted disruption of the blood brain barrier of the patient. In one example, the treatment can utilize an anti-beta amyloid antibody (e.g., Aducanumab, Lecanemab,etc). Other therapeutic agents can also be used to improve the cogitative disorder. In the instance of the therapeutic agent being an anti-beta amyloid antibody (e.g., that target beta amyloid and its constituents), such a beta amyloid antibody treatments (e.g. Aducanumab, Lecanemab, Donanemab)

While the use of neuromodulation for treatment of cognitive disorders shows great promise, selecting an appropriate neuromodulation target and assessing the effectiveness of the selected target remains a challenge. The systems and methods addressed herein utilize a personalized feedback system to efficiently determine the effectiveness of a selected location, allowing a clinician to rapidly adjust the treatment to ensure its effectiveness. To this end, acute feedback, subacute feedback, and chronic feedback can be acquired and evaluated at a machine learning system to determine the efficacy of a given treatment.

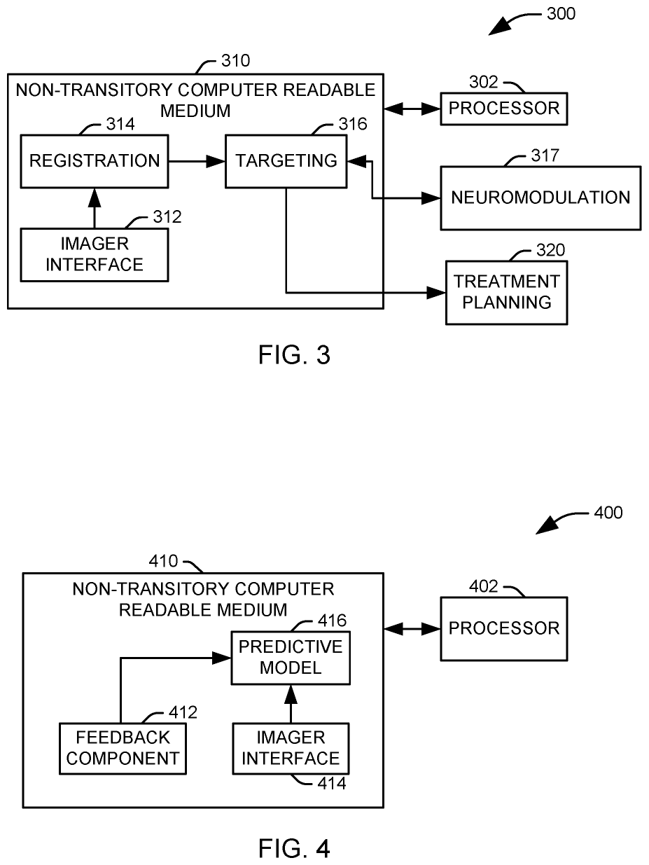

FIG. 3 illustrates a system 300 for targeting neuromodulation for treatment or diagnosis of cognitive disorders to a specific region of the brain of a patient. The system 300 includes a processor 302 and a non-transitory computer readable medium 310 that stores executable instructions for targeting neuromodulation for treatment or diagnosis of cognitive disorders. The executable instructions include an imager interface 312 that receives a first image, representing a structure of the brain, and a second image, representing a connectivity of the brain, for a patient from one or more associated imaging systems (not shown). In one implementation, the first image is a T1 magnetic resonance imaging (MRI) image, and the second image is a diffusion tensor imaging (DTI) image generated using an MRI imager. The imager interface 312 can include appropriate software components for communicating with an imaging system (not shown) or repository of stored images (not shown) over a network via a network interface (not shown) or via a bus connection.

The first image is provided to a registration component 314 that segments the first image into a plurality of subregions of the brain. The identified subregions can include, for example, a frontal pole, a temporal pole, a superior frontal region, a medial orbito-frontal region, a caudal anterior cingulate, a rostral anterior cingulate, an entorhinal region, a parahippocampal region, a peri-calcarine region, a lingual region, a cuneus region, an isthmus region, a pre-cuneus region, a paracentral lobule, and a fusiform region. In one example, the registration component 314 registers the first image to a standard atlas to provide the segmentation. In another implementation, a convolutional neural network, trained on a plurality of annotated image samples, can be used to provide the segmented image. One example of such a system can be found in 3D Whole Brain Segmentation using Spatially Localized Atlas Network Tiles, by Huo et al. (available at https://doi.org/10.48550/arxiv.1903.12152), which is hereby incorporated by reference in its entirety. The registration component 314 can also register the second image with the first image, such that the location of nodes within the connectome within the brain is known.

Each of the segmented first image and the second image can be provided to a targeting component 316 that selects a location and intensity profile for the neuromodulation. It will be appreciated that the segmented first image can be registered with the second image before it is provided to the targeting component. The targeting component 316 generates a connectome of the brain, representing neural connections within the brain, from the second image. A region of interest can be defined within the first image based upon the known subregions, and the location and intensity profile of the neuromodulation within the region of interest can be selected according to the generated connectome. It will be appreciated that the connectome can be determined as a passive connectome, representing the physical connectivity among portions of the brain, or an active connectome, representing the activity induced in portions of the brain in response to energy provided in a specific location.

In one example, the targeting component 316 can operate in conjunction with a neuromodulation system 317 to generate a map of an active connectome of the brain. Specifically, energy can be applied at various locations within the region of interest, and activity within the brain can be determined via an appropriate functional imaging modality. In one implementation, the activity measured within the brain in response to each location is recorded as a result of neuromodulation for that location. It will be appreciated that multiple locations can be impacted when energy is provided, and the detected activity can be attributed to each location, for example, represented as voxels, according to the percentage of energy received at each voxel. Alternatively, the results of multiple measurements can be compared, for example, via solving for one or more n-dimensional linear systems, where n is the number of voxels within the region of interest. Accordingly, the active connectivity associated with each location within the region of interest can be determined.

In one example, determining a target site of the brain that is affected by the cognitive disorder can comprises determining one or more regions of the patient's brain that contains deposits of beta amyloid protein, tau protein, or another biomarker of the cognitive disorder. Determining the presence of such deposits can be performed in a variety of ways but in an aspect, determining the brain regions with these deposits can comprise obtaining at least one positron emission tomography (PET) scan of the patient's brain, obtaining at least one structural or anatomical magnetic resonance imaging (MRI) scan of the patient's brain, merging the at least one PET scan and the at least one MRI scan to create at least one merged scan, and determining the one or more regions of the patient's brain comprising a presence of the beta amyloid protein, the tau protein and/or another biomarker of the cognitive disorder based on the at least one merged scan.

In one implementation, the region of interest can be divided into a set of voxels, and each voxel can be assigned a cost based upon its connection to other regions of the brain in the connectome to form a cost map. For example, a positive cost can represent representing a location within the region of interest that is connected to portions of the brain for which, taken in aggregate, stimulation is not desirable and a negative cost can represent a location within the region of interest that is connected to portions of the brain for which, taken in aggregate, stimulation is desirable. It will be appreciated that this can be reversed to instead create a “utility map,” with positive values representing locations for which stimulation is desirable.

Each location has an associated intensity profile around a reference point, such as a center point, representing an amount of energy provided to the region for a given location of the reference point. In one example, each voxel can be assigned a value normalized by a maximum intensity, and this value can be used to weight the contribution of the voxel to the overall cost associated with the location and intensity profile. An optimization process, such as gradient decent, can be used to search the region of interest for an optimal or near-optimal location and intensity profile, and the resulting location and intensity profile can be provided to a treatment planning system 320 for use in generating a treatment plan for the patient.

FIG. 4 illustrates a system 400 for evaluating the effects of neuromodulation on one or more patients. The system 400 includes a processor 402 and a non-transitory computer readable medium 410 that stores executable instructions for targeting neuromodulation for treatment and diagnosis of compromised cognitive functions. The executable instructions include a feedback component 412 that determines the effectiveness of treatment for a patient according to patient data collected during or after therapy. In one implementation, identified neural activity can be utilized as feedback for determining the success of a treatment of the patient via neuromodulation. This can be done during or immediately after treatment (“acute feedback”), a short time (e.g., five hours to five days) after a treatment (“subacute feedback”) or a longer time (e.g., more than five days after a treatment (“chronic feedback”). The feedback can be acquired, for example via activities measuring a memory and attention of the patient, such as word recall tasks, n-back tasks, flanker tasks, anti-saccade tasks, spatial memory tasks, or other tasks appropriate to evaluating the effects of a given disorder on the patient. For example, to collect acute feedback, the tasks can be presented during or immediately after treatment to determine whether the patient's performance increases, decreases or otherwise changes in response to the treatment. In one example, the patient can be allowed to explore a virtual reality environment and collect items within the environment. The patient is then asked to recount where each item was found within the virtual environment and the relationship of that location to a starting point, testing the patient's ability to recall spatial relationships among the virtual locations. The collected feedback can also include self-reporting from the patient, observations by clinicians, the measured electrical activity, measured biometric parameters, such as heart rate variability and blood pressure, and other relevant parameters.

Acute and subacute feedback from presentation application of a treatment can be obtained, for example, by measuring physiological parameters. In some examples, the physiological parameter can be a response of the patient's autonomic nervous system to neuromodulation and multiple physiological parameters can be measured during any given assessment session. The physiological parameters can be measured via a wearable device such as a ring, watch, or belt or via a smart phone or tablet, for example, in a naturalistic non-clinical setting such as when the patient is at home, work or other non-clinical setting. Exemplary physiological parameters include heart rate, heart rate variability, perspiration, salivation, blood pressure, pupil size, changes in pupil size, eye movements, brain activity, electrodermal activity, body temperature, and blood oxygen saturation level. Table I, above, provides non-limiting examples of physiological parameters that can be measured and exemplary tests to measure the physiological parameters.

As part of the acute and subacute feedback, cognitive parameters can be assessed by a battery of cognitive tests that measure, for example, executive function, decision making, working memory, attention, and fatigue. Table II, above, provides non-limiting examples of cognitive parameters that are gamified and that can be measured and exemplary methods and tests/tasks to measure such cognitive parameters.

These cognitive tests can be administered in a clinical/laboratory setting or in a naturalistic, non-clinical setting such as when the user is at home, work, or other non-clinical setting. A smart device, such as a smartphone, tablet, or smart watch, can facilitate measuring these cognitive parameters in a naturalistic, non-clinical setting. For example, the Erikson Flanker, N-Back and Psychomotor Vigilance Tasks can be taken via an application on a smart phone, tablet, or smart watch.

Behavioral and psychosocial parameters, such as those described in Table III above, can measure the user's functionality, such as the user's movement via wearable devices as well as subjective/self-reporting questionnaires. The subjective/self-reporting questionnaires can be collected in a clinical/laboratory setting or in a naturalistic, non-clinical setting such as when the user is at home, work, or other non-clinical setting. A smart device, such as a smartphone, tablet, or personal computer can be used to administer the subjective/self-reporting questionnaires. Using embedded accelerometers and cameras, these smart devices can also be used to capture the user's movements as well as facial expression analysis to analyze the user's facial expressions that could indicate mood, anxiety, depression, agitation, and fatigue. A wearable or portable device can also be used to measure parameters representing sleep length, circadian rhythms, sleep cycle ratios, sleep depth, a length of a sleep stage, overall sleep length, and heart rate variability.

The type of change of the patient's physiological parameter measurement values after neuromodulation can influence whether further neuromodulation is provided and how the provided neuromodulation should be adjusted. In terms of adjusting therapy in the context of neuromodulation, methods can involve adjusting the parameters or dosing of the neuromodulation such as, for example, the duration, frequency, or intensity of the neuromodulation. When the collected data indicates that the patient's condition has not improved, a method can involve adjusting the neuromodulation so that the neuromodulation is more effective. For example, if the patient was previously having focused ultrasound (FUS) delivered for five minutes during a therapy session, the patient can have the FUS subsequently delivered for twenty minutes during each session or if the patient was having FUS delivered every thirty days, the patient can have FUS subsequently delivered every two weeks. Conversely, if the parameter measurements indicate improvement, the neuromodulation parameters may not need adjustment and subsequent neuromodulation sessions can serve primarily as maintenance sessions or the intensity, frequency or duration of the neuromodulation can be decreased, for example. The above scenarios are only exemplary and are provided to illustrate that the presence and type of change of the patient's physiological parameter measurement values during and after therapy can influence whether the therapy should be adjusted or terminated.

Further, the degree of the patient's physiological, cognitive, psychosocial, or behavioral parameter measurement value during or after therapy can influence the parameters of subsequent neuromodulation. For example, if the specific patient seeking therapy has a physiological, cognitive, psychosocial, or behavioral parameter measurement value during or after treatment that is higher than the average parameter measurement value of the same patient population, the therapy can be more aggressive subsequently. Conversely, if the specific patient's parameter measurement value during or after treatment is lower than the average parameter measurement value of the same patient population, the therapy can be less aggressive subsequently. In other words, the severity or degree of the patient's physiological, cognitive, psychosocial, or behavioral parameter measurement value during or after neuromodulation (as well as baseline values and levels) can correlate to the degree or aggressiveness of future neuromodulation. The above scenarios are only exemplary and are provided to illustrate that the degree of change of the patient's physiological parameter measurement values during and after neuromodulation can influence the parameters of subsequent therapy.

In certain aspects, acute, subacute, and chronic feedback each determined from one or more combinations of a physiological, a cognitive, a psychosocial, and a behavioral parameter of the patient after a treatment. For example, obtaining a measurement of baseline values of one or more combinations of a physiological, a cognitive, a psychosocial, and a behavioral parameter of the patient can be obtained. The patient can then be exposed to neuromodulation such as an initial focused ultrasound signal, an initial deep brain stimulation signal, or an initial transcranial magnetic stimulation signal to a neural target site of the patient. A subsequent measurement can be obtained of resultant values of the one or more combinations of the physiological, the cognitive, the psychosocial, and the behavioral parameter of the patient during or after application of the initial focused ultrasound signal, the initial deep brain stimulation signal, or the initial transcranial magnetic stimulation signal. The resultant values can be compared to the baseline values to determine if the patient's cognitive and/or behavioral functions has improved. The neuromodulation can be adjusted upon a determination that the patient's cognitive and/or behavioral functions has not improved. For example, if it is determined that the neuromodulation was not successful, the neuromodulation can be provided to a different target location.

In the illustrated implementation, chronic feedback can be obtained via imaging of the brain and eye, specifically the retina, optic nerve, and associated vasculature, to observe changes relative to the presence or progression of a cognitive disorder. To this end, the executable instructions can further include an imaging interface 414 that receives a first image, representing a brain of a patient, and a second image, representing an eye of the patient. The first image can be taken, for example, as one or more of a PET scan (including, for example, PET scan using fluorodeoxyglucose (FDG) PET scan, or a PET scan with a radioactive fluorine labeled ligand-linked marker), a gradient recalled echo T2*—weighted imaging (GRE T2*) scan, a, magnetization T1 preparation sequence scan, a T2 weighted fluid attenuation inversion recovery (FLAIR) scan, a susceptibility weighted imaging (SWI) scan, a T1 contrast scan (including, for example, a T1 gadolinium contrast scan), an arterial spin labeling (ASL) scan, and/or a dynamic contrast-enhanced (DCE) MR perfusion scan. In one implementation, the first image is a T1 magnetic resonance imaging (MRI) image, and the second image is an optical coherence tomography (OCT) angiography image. The imaging interface 414 can also receive additional images, for example, from a same or a different type of imager, representing the eye or the brain. The imaging interface 414 can include appropriate software components for communicating with an imaging system (not shown) or repository of stored images (not shown) over a network via a network interface (not shown) or via a bus connection.

In some examples, where the imaging parameter is a PET scan, the scan can be analyzed to determine changes in the concentration of beta-amyloid proteins, tau proteins, and/or other biomarkers of the cognitive disorder based on, for example, previous PET scan(s) or predetermined threshold/baseline values for these biomarkers. The therapy can be delivered or adjusted based on the changes in the concentration. For example, the therapy can be adjusted or delivered upon a detection of a reduction of a concentration or density of a beta-amyloid protein, a tau protein, and/or another biomarker of the cognitive disorder. Alternatively, the therapy can be adjusted or delivered upon a detection of no reduction in the concentration/density of the beta-amyloid protein, the tau protein, and/or another biomarker of the cognitive disorder; or upon a detection of a reduction in the concentration/density of the beta-amyloid protein, the tau protein and/or another biomarker of the cognitive disorder that is insufficient to improve the human patient's cognitive disorder. This determination can be performed acutely, sub-acutely or chronically after providing the neuromodulation. As such, these signals can provide immediate/acute feedback during treatment, sub-acute feedback after minutes/hours/days of providing therapy and chronically after weeks/month of providing therapy.

A predictive model 416 receives a representation of the first image and a representation of the second image and generates a value representing one or more cognitive disorders for the patient. For example, the generated value can be a categorical or continuous value that represents a degree of progression for a specific cognitive disorder or a cognitive disorder generally. In another example, the generated value is a categorical or continuous value that represents a likelihood that the patient will respond to a specific treatment for a cognitive disorder or to treatment of a cognitive disorder generally. In a further example, the generated value is a categorical value that represents an expected best treatment for a cognitive disorder for the patient. The value can be provided to a technician to guide the location and timing of further neuromodulation applied to the patient.

In one example, information gathered across a set of patients with either a general category of cognitive disorders or a specific cognitive disorder can be used to guide the selection of an initial location for applying neuromodulation. For example, if a particular location, defined relative to one or more landmark structures within the brain, has been consistently successful across a set of patients with the same or a similar disorder, that location can be a default location for an initial treatment via neuromodulation. Feedback representing the patient's response to treatment can be gathered as described above, and a new location can be selected for the patient where needed. The new location can be determined from the information gathered for the set of patients. In one implementation, an analogical reasoning system can be used to locate past patients with similar characteristics to a current patient, and locations that were successful in the similar patients can be used. The characteristics used for matching patients with analogous patients can include demographical characteristics, medical histories, measured biometric parameters, such as blood pressure and heart rate variability, observations of the patient in response to cognitive tasks, and measured electrical activity in the brain during and after neuromodulation.

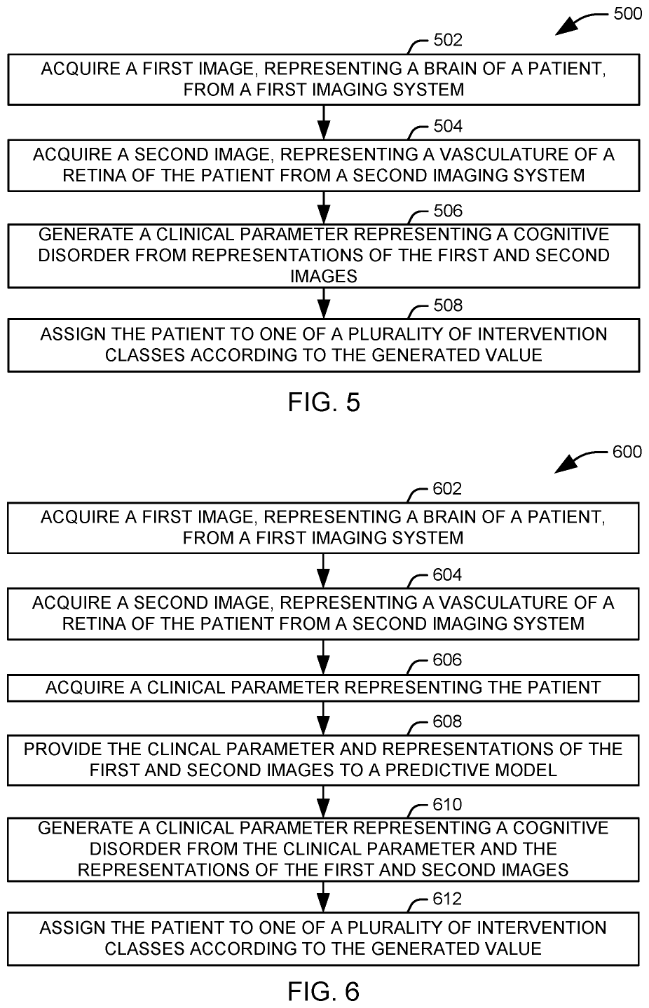

In view of the foregoing structural and functional features described above, an example method will be better appreciated with reference to FIGS. 5-7. While, for purposes of simplicity of explanation, the example methods of FIGS. 5-7 are shown and described as executing serially, it is to be understood and appreciated that the present examples are not limited by the illustrated order, as some actions could in other examples occur in different orders, multiple times and/or concurrently from that shown and described herein. Moreover, it is not necessary that all described actions be performed to implement a method in accordance with the invention.

FIG. 5 illustrates a method 500 for generating a value representing a risk and/or progression of one or more cognitive disorders. At 502, a first image, representing a brain of a patient, is acquired from a first imaging system. In one example, the first imaging system is a magnetic resonance imaging (MRI) imager. Additionally or alternatively, the first image can be acquired via diffusion tensor imaging. In one example, the representation of the first image represents a cortical profile of the brain. In another example, the representation of the first image represents a vasculature of the brain. In a further example, the representation of the first image represents a B-amyloid profile of the brain. In a still further example, the representation of the first image represents a connectivity of the brain. At 504, a second image, representing one of a retina, optic nerve, and associated vasculature of the patient, is acquired from a second imaging system. In one example, the second image is an optical coherence tomography angiography image. A representation of each of the first image and the second image to a machine learning model. In one example, it will be appreciated that the representation of each image can include the chromaticity values associated with each pixel or voxel in the image, such that all or most of the data associated with the image is provided to the machine learning system. Alternatively, a set of numerical features representing the content of each image can be extracted, such as total areas or volumes of structures or tissues of interest. In one example, a pupil of the patient can be imaged to provide an additional parameter representing at least one of eye tracking data, eye movement, pupil size, and a change in pupil size.

At 506, the value is determined at the machine learning model from the representation of the first image and the representation of the second image. In one implementation, a clinical parameter is also provided to the machine learning model for generating the value at the machine learning model. The clinical parameter can be extracted from an electronic health records (EHR) database and represent, for example, a medical history of the patient, a treatment prescribed to the patient, and a measured biometric parameter of a patient, such as values representing a resting heart rate of the patient, a heart rate variability of the patient, sleep quality, and performance metrics on cognitive tests. At 508, the patient is assigned to one of a plurality of intervention classes according to the generated value. In one example, the value can represent a risk of a cognitive disorder, and high-risk patients can be assigned lifestyle changes, such as changes in diet, exercise, and sleep hygiene. In another example, the value can represent a progression of a cognitive disorder, with patients with minimal progression being monitored, patients with intermediate levels of progression treated with neuromodulation, and patients with high levels of progression treated with medication or antibodies in combination with selective disruption of the blood brain barrier, for example, via targeted ultrasound.

Based on the determined intervention class, the therapy can be delivered or adjusted in a variety of ways. Non-limiting ways to deliver or adjust the therapy can at least one or more of providing therapy, not providing therapy, changing a frequency of providing the therapy, adjusting the dose of the neuromodulation, adjusting a timing of applying the neuromodulation, changing a frequency of application of the neuromodulation, changing one or more of the ultrasound delivery device parameters for neuromodulation delivered via focused ultrasound, changing an energy profile of the neuromodulation, changing a location of the therapy from the target site to another site of the human patient's brain. The therapy can be adjusted in other ways as well.

In certain instances, based on the feedback and the determined intervention class, a patient can be determined as a suitable candidate for therapy or not. As such, another example of providing therapy based on the determined intervention class can include providing the therapy if the patient is a suitable candidate for therapy, or foregoing therapy if the patient is not a suitable candidate for therapy.

In one example, the therapy can comprise applying focused ultrasound sonication via an ultrasound delivery device to a target site associated with one or more of attention, executive function, memory, anxiety, behavioral, visual and spatial orientation, sensory processing, and cognition. A non-exhaustive list of potential target sites includes the nucleus basalis of maynert, the ventral capsule/ventral striatum, nucleus accumbens, hippocampus, a thalamic intralaminar nucleus, the pulvinar nucleus, the subthalamic nucleus, other thalamic nuclei, the sub genual cingulate, the fornix, the medial or inferior temporal lobe, the temporal pole, the angular gyrus, the superior or medial frontal lobe, the superior parietal lobe, the precuneus, the supramarginal gyrus, the calcarine sulcus, or combinations thereof. Non-limiting parameters of the initial FUS are Sonication dose, Power (less than 150 W), Sonication Duration (e.g. 0 min-60 min), frequency direction, Repetition Time On/Off (e.g. 5 sec;10 sec), Pulse Duration On/Off (e.g. 100 msec;900 msec), continuous or intermittent, Energy/Minute (e.g. 0 J/min-290 J/min, and Frequency (e.g., 1-3 MHz), number of elements (e.g. 1-1024), and waveshape form. Non-limiting parameters of the initial DBS are frequency (e.g., ˜1 Hz to 10,000 Hz), pulse width (e.g., ˜5 microseconds to ˜1000 microseconds), intensity (e.g. ˜0.1 v or mA to ˜30 v or mA, and waveform shape. Non-limiting parameters of the initial TMS are intensity (e.g., ˜0 to ˜200% resting motor threshold); frequency (e.g., ˜. 01 Hz to ˜30 Hz); type of stimulation (e.g. single, repetitive, and/or patterned); and duration (e.g. ˜1 to ˜90 min).

In one implementation, focused ultrasound treatment is provided with a power between 60 W and 90 W. In another implementation, focused ultrasound treatment is provided with a power between 70 W and 100 W. In a further implementation, focused ultrasound treatment is provided with a power between 40 W and 70 W. In a further implementation, focused ultrasound treatment is provided with a power less than 70 W. In a further implementation, focused ultrasound treatment is provided with a power less than 100 W. In a further implementation, focused ultrasound treatment is provided with a power less than 150 W. In a further implementation, focused ultrasound treatment is provided with a power between 40 W and 100 W. In a further implementation, focused ultrasound treatment is provided with a power between 50 W and 90 W. In a further implementation, focused ultrasound treatment is provided with a power between 60 W and 100 W. In a further implementation, focused ultrasound treatment is provided with a power between 70 W and 120 W.

In one implementation, focused ultrasound treatment is provided with a pulse width between 10 ms and 100 ms. In another implementation, focused ultrasound treatment is provided with a pulse width between 10 ms and 50 ms. In a further implementation, focused ultrasound treatment is provided with a pulse width between 10 ms and 200 ms. In a further implementation, focused ultrasound treatment is provided with a pulse width between 50 ms and 200 ms. In a further implementation, focused ultrasound treatment is provided with a pulse width between 100 ms and 200 ms. In one implementation, a session of focused ultrasound treatment lasts between three minutes and seven minutes. In another implementation, a session of focused ultrasound treatment lasts between five minutes and twenty minutes. In a further implementation, a session of focused ultrasound treatment lasts between three minutes and ten minutes. In a further implementation, a session of focused ultrasound treatment lasts between five minutes and twenty minutes. In a further implementation, a session of focused ultrasound treatment lasts between ten minutes and twenty minutes. In a further implementation, a session of focused ultrasound treatment lasts between ten minutes and thirty minutes. In a further implementation, a session of focused ultrasound treatment lasts between ten minutes and sixty minutes.

In one implementation, focused ultrasound treatment is provided with a frequency between 0.02 MHz and 0.1 MHz. In another implementation, focused ultrasound treatment is provided with a frequency between 0.2 MHz and 0.3 MHz. In a further implementation, focused ultrasound treatment is provided with a frequency between 0.4 MHz and 0.6 MHz. In a further implementation, focused ultrasound treatment is provided with a frequency between 0.4 MHz and 0.8 MHz. In a further implementation, focused ultrasound treatment is provided with a frequency between 0.7 MHz and 1 MHz. In a further implementation, focused ultrasound treatment is provided with a frequency between 1 MHz and 2 MHZ. In a further implementation, focused ultrasound treatment is provided with a frequency between 2 MHz and 3 MHz. In a further implementation, focused ultrasound treatment is provided with a frequency between 0.1 MHz and 3 MHz.

FIG. 6 illustrates another method 600 for generating a value representing a risk and/or progression of one or more cognitive disorders. At 602, a first image, representing a brain of a patient, is acquired from a first imaging system. At 604, a second image, representing one of a retina, an optic nerve, and an associated vasculature of the patient, is acquired from a second imaging system. At 606, a clinical parameter representing the patient is acquired. In one example, the clinical parameter is received from a device worn by the patient. In another example, the clinical parameter is retrieved from an electronic health records database. In still another example, the clinical parameter represents one of eye tracking data, eye movement, pupil size, and a change in pupil size. The clinical parameter and a representation of each of the first image and the second image are provided to a machine learning model at 608. At 610, the value is generated at the machine learning model from the representation of the first image, the representation of the second image, and the clinical parameter. At 612, the patient is assigned to one of a plurality of intervention classes according to the generated value.

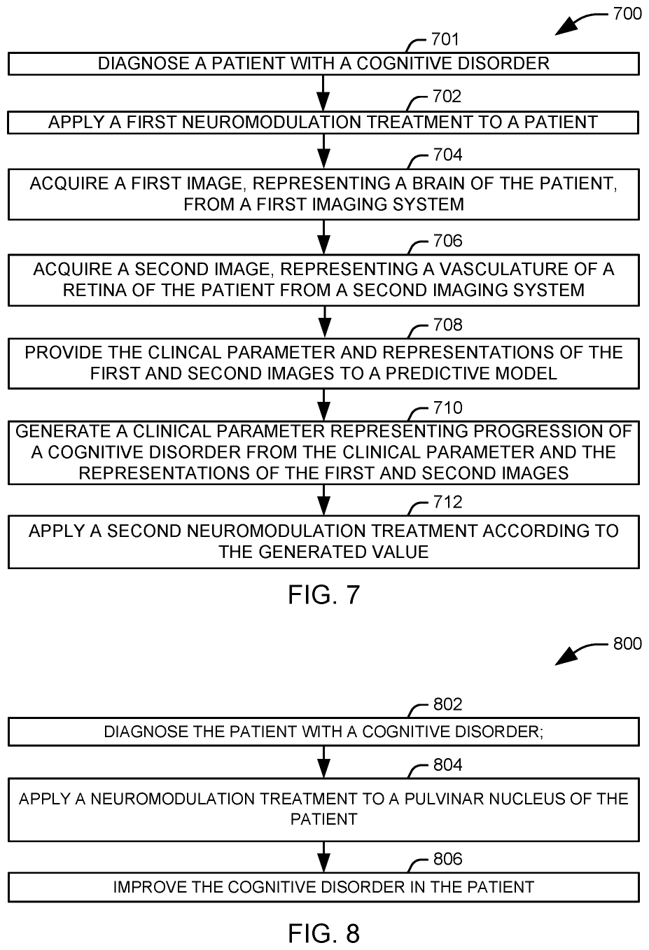

FIG. 7 illustrates a method 700 for obtaining chronic feedback for a neuromodulation treatment. At 701, a patient is diagnosed with a cognitive disorder. In one example, the patient can be diagnosed via one of the methods of FIGS. 5 and 6. At 702, a first neuromodulation treatment is applied to a patient at a location of interest within the brain. At 704, a first image, representing a brain of a patient, is acquired from a first imaging system. In one example, the first imaging system is a magnetic resonance imaging (MRI) imager. Additionally or alternatively, the first image can be acquired via diffusion tensor imaging. In one example, the representation of the first image represents a cortical profile of the brain. In another example, the representation of the first image represents a vasculature of the brain. In a further example, the representation of the first image represents a B-amyloid profile of the brain. In a still further example, the representation of the first image represents a connectivity of the brain. At 706, a second image, representing one of a retina, optic nerve, and associated vasculature of the patient, is acquired from a second imaging system. In one example, the second image is an optical coherence tomography angiography image. A representation of each of the first image and the second image to a machine learning model. In one example, it will be appreciated that the representation of each image can include the chromaticity values associated with each pixel or voxel in the image, such that all or most of the data associated with the image is provided to the machine learning system. Alternatively, a set of numerical features representing the content of each image can be extracted, such as total areas or volumes of structures or tissues of interest.

At 708, a representation of each of the first image and the second image is provided to a machine learning model. In one example, a parameter representing at least one of eye tracking data, eye movement, pupil size, and a change in pupil size. Can also be provided to the machine learning model. At 710, a value representing progression of a cognitive disorder is generated at the machine learning model from the representation of the first image and the representation of the second image. For example, the generated value can be a continuous or categorical parameter representing a progression of a cognitive disorder, a categorical parameter representing a location for further neuromodulation, or a categorical or continuous parameter representing a timing for further neuromodulation. At 712, a second neuromodulation treatment to the patient according to the generated value. For example, a location or timing of the second neuromodulation treatment can be selected according to the generated value.

In accordance with another aspect of the invention, a method 800 of improving a cognitive disorder in a patient suffering therefrom is provided as illustrated in FIG. 8. The method includes diagnosing the patient with a cognitive disorder 802, applying a neuromodulation treatment to a pulvinar nucleus of the patient 804, and improving the cognitive disorder in the patient 806. The neuromodulation technique can comprise focused ultrasound treatment, deep brain stimulation, or transcranial magnetic stimulation. The cognitive disorder can be improved in many ways as described above including improving visual attention function, a visio-spatial orientation of the patient, or both. In certain aspects, a method to improve cognitive function can further include stimulating sited in conjunction with/in addition to the pulvinar nucleus such as, for example, the nucleus basalis of maynert, the ventral capsule/ventral striatum, the nucleus accumbens, the hippocampus, a thalamic intralaminar nucleus, the subthalamic nucleus, the sub genual cingulate, the fornix, the medial or inferior temporal lobe, the temporal pole, the angular gyrus, the superior or medial frontal lobe, the superior parietal lobe, the precuneus, the supramarginal gyrus, the calcarine sulcus, or combinations thereof. In certain aspects and as described above, the method can include allowing the patient to explore a virtual reality environment and identify a plurality of items within the virtual reality environment, instructing the patient to recount locations of the plurality of items in the virtual reality environment and the relationship of a location of each of the plurality of items relative to a starting point, determining the patient's ability to recall a spatial relationship among the locations of the plurality of items, and adjusting application of the neuromodulation treatment based on the determination.

In another aspect a method of improving a cognitive disorder in a patient suffering therefrom comprises diagnosing the patient with a cognitive disorder, acquiring a clinical parameter associated with the patient, applying a neuromodulation treatment or adjusting application of a neuromodulation treatment to a pulvinar nucleus of the patient based on the clinical parameter, and improving the cognitive disorder in the patient. The clinical parameter can include any suitable clinical parameter disclosed above including visual performance, visio-spatial performance, perceptual motor tasks, working memory, or combinations thereof. It should be noted that aspects of the present invention directed to targeting the pulvinar nucleus can be used with other aspects of the invention disclosed above.

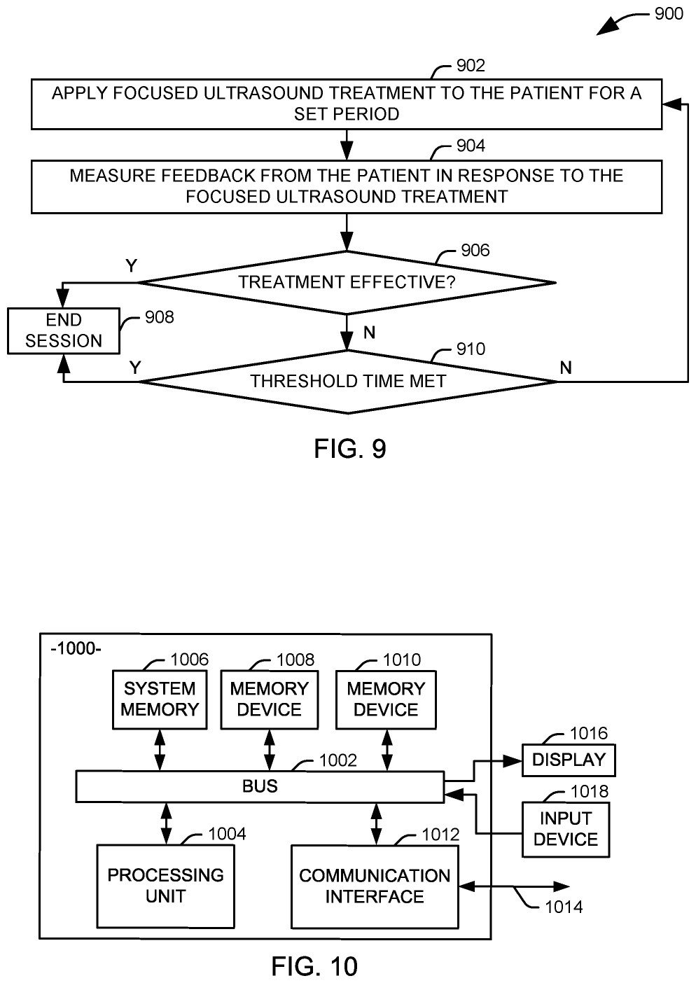

FIG. 9 illustrates a method 900 for providing ultrasound treatment. At 902, focused ultrasound treatment is applied to the selected location for a set period of time, for example, a period of five minutes, and feedback from the patient in response to the applied focused ultrasound treatment is measured at 904. The feedback can utilize various cognitive tests, particularly visuospatial tests, and tests of visual focus and pupil response, can include, for example, the N-Back Task, episodic memory tasks, spatial navigation tasks, recall of word lists, picture naming, anti-saccade tasks, and sustained attention tasks. In one example of a spatial navigation test, the patient can be allowed to explore a virtual reality environment and collect items within the environment. The patient is then asked to recount where each item was found within the virtual environment and the relationship of that location to a starting point, testing the patient's ability to recall spatial relationships among the virtual locations.

At 906, an effectiveness of the focused ultrasound treatment is determined according to the measured feedback. This can be done, for example, either by a rule-based approach, in which the results of one or more cognitive test are compared to various thresholds, or by providing the test results to a predictive model to classify the patient into one of a plurality of classes representing the effectiveness of the treatment. If the modulation is determined to be effective (Y), the session of neuromodulation is ended at 908. If not (N), the method advances to 910, where it is determined if a total time for the session of neuromodulation has met a threshold time. A threshold time is considered met when another set period of neuromodulation would exceed the threshold time. In one example, the threshold time is thirty minutes. In another example, the threshold time is an hour. If the threshold time has been met (Y), the session of neuromodulation is ended at 908. If the threshold has not been met (N), the method returns to 902 to perform another round of neuromodulation for the set period.

FIG. 10 is a schematic block diagram illustrating an exemplary system 1000 of hardware components capable of implementing examples of the systems and methods disclosed in FIGS. 1-9, such as the system for diagnosis and monitoring of cognitive disorders illustrated in FIG. 1. The system 1000 can include various systems and subsystems. The system 1000 can be any of personal computer, a laptop computer, a workstation, a computer system, an appliance, an application-specific integrated circuit (ASIC), a server, a server blade center, or a server farm.

The system 1000 can includes a system bus 1002, a processing unit 1004, a system memory 1006, memory devices 1008 and 1010, a communication interface 1012 (e.g., a network interface), a communication link 1014, a display 1016 (e.g., a video screen), and an input device 1018 (e.g., a keyboard and/or a mouse). The system bus 1002 can be in communication with the processing unit 1004 and the system memory 1006. The additional memory devices 1008 and 1010, such as a hard disk drive, server, stand-alone database, or other non-volatile memory, can also be in communication with the system bus 1002. The system bus 1002 interconnects the processing unit 1004, the memory devices 1006-1010, the communication interface 1012, the display 1016, and the input device 1018. In some examples, the system bus 1002 also interconnects an additional port (not shown), such as a universal serial bus (USB) port.

The system 1000 could be implemented in a computing cloud. In such a situation, features of the system 1000, such as the processing unit 1004, the communication interface 1012, and the memory devices 1008 and 1010 could be representative of a single instance of hardware or multiple instances of hardware with applications executing across the multiple of instances (i.e., distributed) of hardware (e.g., computers, routers, memory, processors, or a combination thereof). Alternatively, the system 1000 could be implemented on a single dedicated server.

The processing unit 1004 can be a computing device and can include an application-specific integrated circuit (ASIC). The processing unit 1004 executes a set of instructions to implement the operations of examples disclosed herein. The processing unit can include a processing core. The additional memory devices 1006, 1008, and 1010 can store data, programs, instructions, database queries in text or compiled form, and any other information that can be needed to operate a computer. The memories 1006, 1008, and 1010 can be implemented as computer-readable media (integrated or removable) such as a memory card, disk drive, compact disk (CD), or server accessible over a network. In certain examples, the memories 1006, 1008 and 1010 can comprise text, images, video, and/or audio, portions of which can be available in formats comprehensible to human beings.

Additionally or alternatively, the system 1000 can access an external data source or query source through the communication interface 1012, which can communicate with the system bus 1002 and the communication link 1014. In operation, the system 1000 can be used to implement one or more parts of a system in accordance with the present invention. Computer executable logic for implementing the quality assurance system resides on one or more of the system memory 1006, and the memory devices 1008, 1010 in accordance with certain examples. The processing unit 1004 executes one or more computer executable instructions originating from the system memory 1006 and the memory devices 1008 and 1010. It will be appreciated that a computer readable medium can include multiple computer readable media each operatively connected to the processing unit.

Implementation of the techniques, blocks, steps, and means described above can be done in various ways. For example, these techniques, blocks, steps, and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof. Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, and volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine-readable mediums for storing information. The terms “computer readable medium” and “machine readable medium” includes, but is not limited to portable or fixed storage devices, optical storage devices, wireless channels, and/or various other storage mediums capable of storing that contain or carry instruction(s) and/or data. It will be appreciated that a “computer readable medium” or “machine readable medium” can include multiple media each operatively connected to a processing unit.

What have been described above are examples. It is, of course, not possible to describe every conceivable combination of components or methodologies, but one of ordinary skill in the art will recognize that many further combinations and permutations are possible. Accordingly, the disclosure is intended to embrace all such alterations, modifications, and variations that fall within the scope of this application, including the appended claims. As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

In view of the foregoing, the following is claimed:

1. A method of improving a cognitive disorder in a patient suffering therefrom comprising:
diagnosing the patient with the cognitive disorder;
applying a neuromodulation treatment to a pulvinar nucleus of the patient; and
improving the cognitive disorder in the patient.

2. The method of claim 1, wherein the neuromodulation treatment comprises focused ultrasound treatment.

3. The method of claim 1, wherein the neuromodulation treatment comprises deep brain stimulation.

4. The method of claim 1, wherein the neuromodulation treatment comprises transcranial magnetic stimulation.

5. The method of claim 1, wherein improving the cognitive disorder comprises improving a visual attention function, a visio-spatial orientation, or both of the patient.

6. The method of claim 1, further comprising:
applying a neuromodulation treatment to a nucleus basalis of maynert, a ventral capsule/ventral striatum, a nucleus accumbens, a hippocampus, a thalamic intralaminar nucleus, a subthalamic nucleus, a sub genual cingulate, a fornix, a medial or inferior temporal lobe, a temporal pole, an angular gyrus, a superior or medial frontal lobe, a superior parietal lobe, a precuneus, a supramarginal gyrus, a calcarine sulcus, or combinations thereof.

7. The method of claim 1, further comprising:
allowing the patient to explore a virtual reality environment and identify a plurality of items within the virtual reality environment;
instructing the patient to recount locations of the plurality of items in the virtual reality environment and the relationship of a location of each of the plurality of items relative to a starting point; and
determining the patient's ability to recall a spatial relationship among the locations of the plurality of items; and
adjusting application of the neuromodulation treatment based on the determination.

8. A method of improving a cognitive disorder in a patient suffering therefrom comprising:
diagnosing the patient with a cognitive disorder;
acquiring a clinical parameter associated with the patient;
applying a neuromodulation treatment or adjusting application of a neuromodulation treatment to a pulvinar nucleus of the patient based on the clinical parameter; and
improving the cognitive disorder in the patient.

9. The method of claim 8, wherein the neuromodulation treatment is focused ultrasound.

10. The method of claim 8, wherein the clinical parameter is visual performance, visio-spatial performance, perceptual motor tasks, working memory, or combinations thereof.
